(19) United States
(12) Patent Application Publication
(10) Pub. No.: US 2026/0265108 A1
(43) Pub. Date: Sep. 10, 2026

(54) ANAEROBIC DIGESTER MONITORING SYSTEM AND METHOD FOR BIOLOGICAL CONTROL

(71) Applicant: Valkyrie Analytics, Boise, ID (US)

(72) Inventor: William Robert Charles Charlton, Boise, ID (US)

(21) Appl. No.: 19/165,290

(22) PCT Filed: Mar. 13, 2024
(86) PCT No.: PCT/US2024/019675

§ 371 Date: Sep. 15, 2025

Related U.S. Application Data
(60) Provisional application No. 63/490,165, filed on Mar. 14, 2023.

(51) Int. Cl. C02F 11/04; B09B 3/65; B09B 101/70; G01N 21/359; G01N 21/84; G01N 21/85

(52) U.S. Cl. CPC C02F 11/04; B09B 3/65; G01N 21/359; G01N 21/85; B09B 2101/70; C02F 2209/005; G01N 2021/8411

(57) ABSTRACT

An anaerobic digester system and method of control are provided. The digester system comprises a main reactor, an influent line in fluid communication with the main reactor, an effluent line in fluid communication with the main reactor, and a monitoring system. The monitoring system including at least one sensor in fluid communication with the influent line or the effluent line. The sensor is configured to determine one or more values associated with one or more physical and chemical parameters of a fluid entering or leaving the reaction. The sensor is communicatively coupled to an analyzer, which is communicatively coupled to a server. The server is configured to receive the one or more determined valves, interpret the one or more determined values, and transmit the one or more determined vales to an interface configured to display the one or more determined values.

### CROSS-REFERENCE TO RELATED APPLICATIONS

This application claims the benefit of U.S. Provisional Patent Application No. 63/490,165, filed Mar. 14, 2023, entitled ANAEROBIC DIGESTER MONITORING SYSTEM AND METHOD FOR BIOLOGICAL CONTROL, the entire contents of which are incorporated herein by reference.

### TECHNICAL FIELD

The present disclosure generally describes control systems and methods relating to biological monitoring for an anaerobic digester system.

### BACKGROUND

Anaerobic digester systems, commonly referred to as biogas digesters, are used in industrial, commercial, and farming settings to decompose organic matter under controlled conditions. Organic matter, such as manure, food waste, sewage, agricultural waste, and the like, can be fed into a digester reactor containing microorganisms. The microorganisms can break the organic matter down into byproducts such as biogas and liquid and solid digestate. The biogas can be collected and burned as fuel, and the digestate can be used as fertilizer. Thus, anaerobic digester systems are well suited for rural environments where waste plants and power generation facilities are not easily accessible.

Anaerobic digester systems may be controlled by monitoring and adjusting feed flow rates, gas flow rates, digestate flow rates, temperature, pH values, and one or more biological parameters. Biological parameters can include physical and chemical indicators such as total solids (TS), volatile solids (VS), chemical oxygen demand (COD), biochemical oxygen demand (BOD), total organic carbon (COD), Total Kjeldahl Nitrogen (TKN), volatile fatty acids (VFA)—also referred to as volatile organic acids (FOS), acetic acid, fat, oil, and grease (FOG), fatty acids (FA), alkalinity, pH value, temperature, total suspended solids (TSS), total dissolved solids (TDS), hydrogen sulfide (H₂S), phosphorous (P₄), potassium (K), carbon dioxide (CO₂), oxygen (O₂), methane (CH₄), nitrogen (N₂), potassium oxide (K₂O), phosphorus pentoxide (P₂O₅), water flow rates, energy levels, organic loading, and relationships between these parameters.

Conventional digester monitoring systems only monitor a couple of these biological parameters. Moreover, even if additional biological parameters are monitored, they are typically determined by collecting field samples to be processed in a laboratory. For example, conventional monitoring systems determine TSS, BOD, VS, and VFA through wet lab analysis. Thus, it can be difficult to diagnose upsets in digester performance because some parameters impacting operation may not be known, and there can be a significant time lag between real-time operation and the monitored data.

Therefore, there is a need for an anaerobic digester system, including an in-line monitoring system, for reliably measuring multiple biological parameters in near real-time so that the anaerobic digester system can be operated to reduce upsets.

### SUMMARY

Provided herein are anaerobic digester systems and methods for monitoring an anaerobic digester system. The systems and methods may, for example, provide for the ability to monitor biological parameters using one or more in-line sensors and, in particular, may overcome the shortcomings related to traditional monitoring systems.

For example, provided herein is an anaerobic digester monitoring system, comprising a first sensor in fluid communication with a first fluid line of an anaerobic digester system, wherein the first sensor is designed to detect one or more values associated with one or more physical or chemical parameters of a first fluid in the first fluid line, wherein the first sensor is communicatively coupled to an analyzer designed to transmit the one or more values to a server including a controller, wherein the controller is designed to determine whether the one or more values are within a target range, and wherein the server is communicatively coupled to an interface configured to display the one or more values and provide an indication that the one or more values are outside of the target range.

The first sensor can be provided, for example, in the form of a near infrared (NIR) sensor.

Optionally, the anaerobic digester monitoring system can further comprise a second sensor in fluid communication with a second fluid line of the anaerobic digester system, wherein the second sensor is configured to detect one or more values associated with one or more physical or chemical parameters of a second fluid in the second fluid line.

Optionally, the first fluid line can be a feed line. The second fluid line can be a digestate line.

The first sensor and/or the second sensor are preferably capable of monitoring a plurality of physical or chemical properties. For example, the one or more physical or chemical properties detected by the first sensor and/or the second sensor may comprise one or more properties selected from the group consisting of total solids (TS), volatile solids (VS), chemical oxygen demand (COD), biochemical oxygen demand (BOD), total organic carbon (COD), Total Kjeldahl Nitrogen (TKN), volatile fatty acids (VFA), acetic acid, fat, oil, and grease (FOG), fatty acids (FA), alkalinity, pH value, temperature, total suspended solids (TSS), total dissolved solids (TDS), hydrogen sulfide (H₂S), phosphorous (P₄), potassium (K), potassium oxide (K₂O), phosphorus pentoxide (P₂O₅), water flow rates, energy levels, and organic loading.

Further provided is an anaerobic digester system, comprising: a main reactor; an influent line in fluid communication with the main reactor; an effluent line in fluid communication with the main reactor; and a monitoring system comprising a first sensor in fluid communication with the influent line, the first sensor configured to detect one or more values associated with one or more physical or chemical parameters of a fluid in the influent line; and an analyzer communicatively coupled to the first sensor and a server, wherein the server is configured to receive the one or more detected valves; interpret the one or more detected values; and transmit the one or more detected vales to an interface configured to display the one or more detected values.

In some instances, the server is designed to determine whether each value of the one or more values is out of compliance with a target range.

For example, the interface may be designed to display an alert in response to the controller determining that at least one value of the one or more values is out of compliance with the target range.

Optionally, the controller may be designed to determine a corrective action designed to bring the one or more out of compliance values into compliance with the target range.

Optionally, the controller may be designed to automatically implement the corrective action.

In some instances, the controller is designed to store a plurality of historical values associated with the one or more physical or chemical parameters in a memory, determine a trend based on the plurality of historical values, determine a trend average of the trend, determine a target range based on the trend average, and determine whether the one or more values are greater than or less than the target range.

Still further provided is a method for controlling an anaerobic digester system. The method comprises detecting one or more values associated with one or more physical or chemical parameters of a first fluid line of the anaerobic digester system with a first sensor, interpreting the one or more values, and transmitting the one or more values to an interface designed to display the one or more values.

Optionally, the method further comprises detecting one or more values associated with one or more physical or chemical parameters of a second fluid line of the anaerobic digester system with a second sensor.

For example, the method may further comprise determining whether each value of the one or more values is within a target range.

The method may, for example, comprise displaying an alert on the interface in response to determining at least one value of the one or more values is not within the target range.

Optionally, the method may further comprise determining a corrective action designed to bring the at least one value within the target range.

The method may comprise storing a plurality of historical values associated with one or more physical or chemical parameters in a memory of a controller, and determining a trend based on the plurality of historical values.

For example, the method may comprise determining a trend average of the trend, and determining a trend threshold range based at least in part on the trend average.

The method may further comprise determining whether at least one value of the one or more values is within the trend threshold range.

### DESCRIPTION OF THE DRAWINGS

Examples are described with reference to the following drawing figures. The same numbers are used throughout the figures to reference features and components.

FIG. 1 is a schematic block diagram of a portion of an anaerobic digester system;

FIG. 2 is a schematic block diagram of a monitoring system including two sensor locations in an anaerobic digester system;

FIG. 3 illustrates a sensor according to an embodiment;

FIG. 4 is an isometric view of a sensor installed in a bypass of an anaerobic digester system;

FIGS. 5A-5B are an isometric and an exploded view, respectively, of a gasket mounting for a sensor according to an embodiment;

FIG. 6 is a side plan view of a gasket mounting for a sensor according to an embodiment;

FIG. 7 illustrates a monitoring system control interface;

FIG. 8 illustrates a method of controlling the anaerobic digester system of FIG. 1;

FIG. 9 is a graph showing energy production for an anaerobic digester before and after the installation of an in-line monitoring system;

FIG. 10 is a graph showing TOC and TKN levels for an anaerobic digester after the installation of an in-line monitoring system;

FIG. 11 is a graph showing acetic acid and total VFA levels for an anaerobic digester after the installation of an in-line monitoring system;

FIG. 12 is a graph showing alkalinity levels for an anaerobic digester after the installation of an in-line monitoring system; and

FIG. 13 is a graph showing COD levels for an anaerobic digester after the installation of an in-line monitoring system.

### DETAILED DESCRIPTION

Before any embodiments of the invention are explained in detail, it is to be understood that the invention is not limited in its application to the details of construction and the arrangement of components set forth in the following description or illustrated in the following drawings. The system is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” “controlled,” “coupled,” and “communicated” and variations thereof are used broadly and encompass both direct and indirect mountings, connections, supports, controls, and couplings. Further, “connected” and “coupled” are not restricted to physical or mechanical connections or couplings and can also include electrically and communicatively coupled configurations in addition to other forms of connections or couplings.

The following discussion is presented to enable a person skilled in the art to make and use embodiments of the system. Various modifications to the illustrated embodiments will be readily apparent to those skilled in the art, and the generic principles herein can be applied to other embodiments and applications without departing from embodiments of the system. Thus, embodiments of the invention are not intended to be limited to embodiments shown but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the system. Skilled artisans will recognize the examples provided herein have many useful alternatives and fall within the scope of embodiments of the invention.

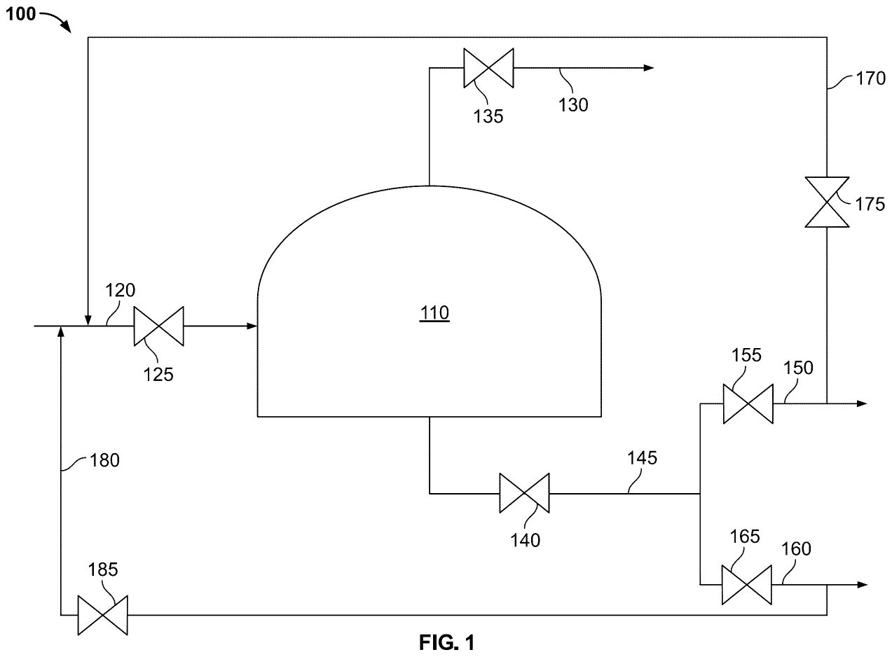

Turning to FIG. 1, a schematic block diagram of a portion of an anaerobic digester system 100 is shown. As seen in FIG. 1, the system 100 can include a main reactor 110, a feed line 120 for transporting organic matter to the main reactor 110, a gas product line 130, and a digestate product line 140, including a liquid product line 150 and a solids product line 160.

The feed line 120 can transport organic matter such as manure, food waste, sewage, agricultural waste, and other biodegradable waste to the main reactor 110 for processing. The feed line 120 can include a first valve 125 designed to control a flow of the organic matter through the feed line 120. It is to be understood that the feed line 120 can include additional components such as a grinder, a mixer, a hopper, a pump, or any other component known in the art. The organic matter may be mixed with a fluid such as water to create a slurry prior to entering the main reactor 110.

The main reactor 110 can be provided in the form of an airtight tank, dome, or other similar structure. The main reactor can include microorganisms capable of breaking down the organic matter into gas, liquid, and solids. The main reactor 110 can include an aeration system, a mixing system, a heater, a digester tank, a gas tank, and any other component known in the art.

As the organic matter is broken down, biogas and digestate is formed. The biogas can be removed from the main reactor 110 through a gas product line 130. The gas product line can be positioned on or near a top area of the main reactor 110. The gas product line 130 can be collected for fuel and/or sent to a scrubber or gas cleaner system for further processing. Accordingly, the gas product line 130 can include a second valve 135 designed to control the flow of the gas through the gas product line 130.

The digestate can be removed through a digestate product line 140 located at or near a bottom of the main reactor 110. The digestate product line 140 can include a third valve 145 designed to control a flow of the digestate through the digestate product line 140. The digestate can be separated into a liquid digestate and a solids digestate, which are removed from the system 100 through a liquid product line 150 and a solids product line 160, respectively. The take-offs for each of the liquid product line 150 and the solids product line 160 can be downstream of the third valve 145. Thus, the liquid product line 150 can include a fourth valve 155 designed to control a flow of the liquid digestate through the liquid product line 150. Similarly, the solid product line 160 can include a fifth valve 165 designed to control a flow of the solid digestate through the solid product line 160.

The liquid and/or solid digestate can be used as fertilizer and/or recycled back through the system 100 (i.e., the liquid and solid digestate can be added to the incoming organic matter to create the slurry). Thus, in some instances, the system 100 can include a first recycle line 170 between and in fluid communication with the liquid product line 150 and feed line 120. Accordingly, the first recycle line 170 can be designed to deliver some or all of the liquid digestate back to the product line 120. Further, the first recycle line 170 can include a sixth valve 175 designed to control a flow of the recycled liquid digestate through the first recycle line 170.

Similarly, the system 100 can include a second recycle line 180 between and in fluid communication with the solid product line 160 and feed line 120. Accordingly, the second recycle line 180 can be designed to deliver some or all of the solid digestate back to the product line 120. Further, the second recycle line 180 can include a seventh valve 185 designed to control a flow of the recycled solid digestate through the second recycle line 180.

It is to be understood that although a single main reactor 110 is shown, the system 100 can include one or more reactors. For example, the system 100 can include a primary digester and a secondary digestor. The primary and the secondary digestors may have the same configuration or may have different configurations.

During operation, the performance of the digester can be measured by the rate of biogas generation. The rate of biogas generation can be dependent on the chemical and physical characteristics of the organic matter fed into the main reactor 110. Further, retention time, organic loading rate (OLR), temperature, and VS, are several common parameters that are monitored to determine the efficiency of the digester. Additionally, VFA, OLR, CH₄ concentration, pH value, alkalinity, the ratio between VFAs and alkalinity, and other biological parameters can provide early warning signs that the main reactor 110 is not operating in a steady state and that an upset such as foaming, can occur.

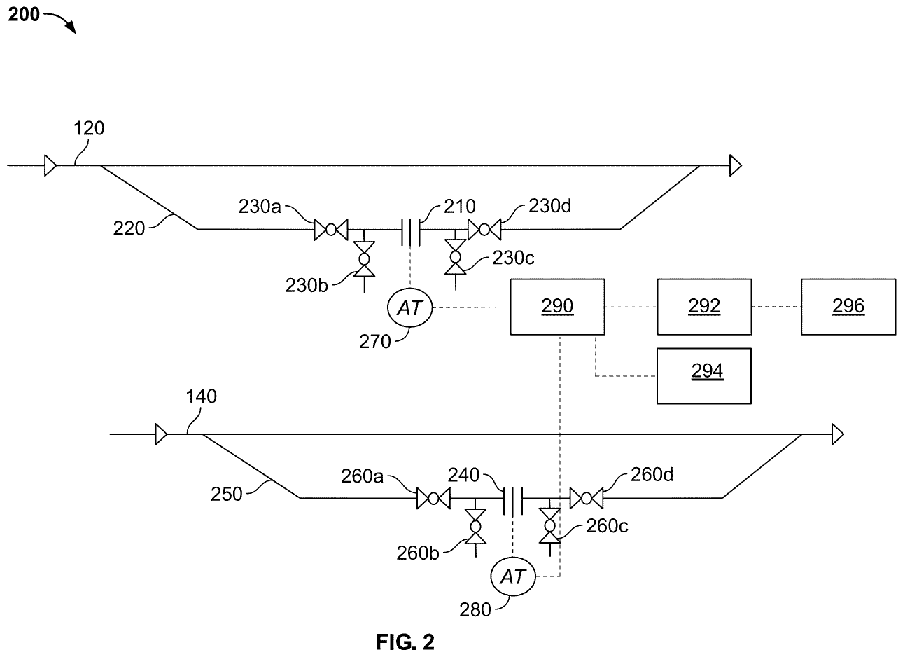

Turning to FIG. 2, a schematic block diagram of an in-line monitoring system 200 is shown. The monitoring system 200 can include a first in-line sensor 210 positioned in a first bypass line 220 of the feed line 120 of FIG. 1. The first bypass line 220 can include one or more valves to control the flow of the organic matter through the first bypass line 220. As shown, the first bypass line 220 includes a first valve 230a and a second valve 230b upstream of the first sensor 210, and a third valve 230c, and a fourth valve 230d downstream of the first sensor 210. However, it is to be understood that the first bypass line 220 can include more or fewer valves.

Similarly, the monitoring system 200 can include a second in-line sensor 240 positioned in a second bypass line 250 of the digestate product line 140 of FIG. 1. The second bypass line 250 can include one or more valves to control the flow of the organic matter through the first bypass line 220. As shown, the second bypass line 250 includes a fifth valve 260a and a sixth valve 260b upstream of the second sensor 240, a seventh valve 260c, and an eighth valve 260d downstream of the second sensor 240. However, it is to be understood that the second bypass line 250 can include more or fewer valves.

Each of the first sensor 210 and the second sensor 240 can be communicatively coupled to a first analyzer 270 and a second analyzer 280, respectively. The first analyzer 270 and the second analyzer 280 can each be communicatively coupled to a server 290.

The server 290 can be provided in the form of a local server, such as a local installation box, or a remote server. The server 290 can be designed to receive data from the first analyzer 270 and/or the second analyzer 280. Further, the server 290 can be designed to transmit data to an operating PC 292 and/or a controller 296. Additionally, a power supply 294 can be designed to supply power to one or more electrical components of the in-line monitoring system 200 (e.g., the first sensor 210, the second sensor 240, the first analyzer 270, the second analyzer 280, the server 290, the operating PC 292, and/or the controller 296).

It is to be understood that the monitoring system 200 can include one or more networks and various communication processes and connections may be implemented to work in conjunction with, or independent from, one or more servers (e.g., the server 290) and/or networks associated with each of the components of the system 100 (i.e., pumps, valves, sensors, probes, etc.)

The network can be provided in the form of a network interface, a local network, or other communication connections. One skilled in the art will recognize that a communication connection can transmit and receive data using a plurality of communication protocols, including but not limited to: wired, wireless, Bluetooth, cellular, satellite, GPS, RS-485, RF, MODBUS, CAN, CANBUS, DeviceNet, ControlNet, Ethernet TCP/IP, RS-232, Universal Serial Bus (USB), Firewire, Thread, proprietary protocol(s), or other known communication protocol(s) as applicable.

In some embodiments, the network is located proximate to one or more components of the anaerobic system. For example, the network may be a local area network (“LAN”). Alternatively, or in addition to, the network can include the Internet, intranets, extranets, wide area networks (“WANs”), wired networks, wireless networks, cloud networks, Ethernet networks, a combination of two or more networks, and other suitable networks.

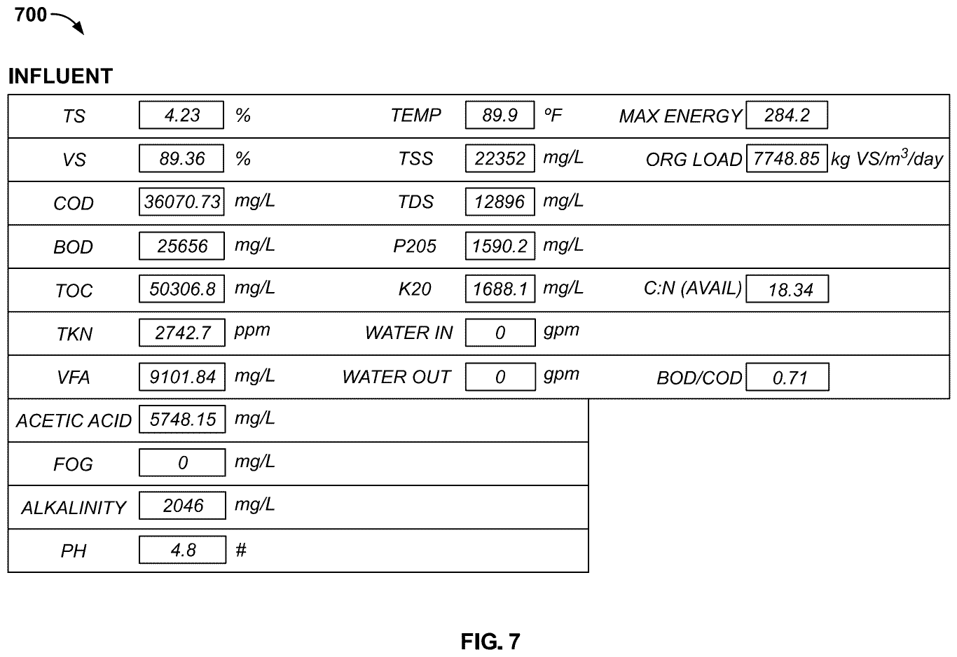

As discussed in more detail below (see FIG. 7), the operating PC 292 can be configured to display one or more values detected by the first sensor 210 and/or the second sensor 240. The operating PC 292 can be communicatively coupled to a controller 296.

The controller 296 can be provided in the form of a program logic controller (PLC) data integration unit. The controller 296 can be communicatively coupled to one or more components of an anaerobic digester system, such as the system 100 of FIG. 1, and be configured to adjust one or more operating parameters of the system 100. For example, the controller can be configured to adjust a flow rate via the first valve 125 through the feed line 120 in response to a value detected by the first sensor 210 and/or the second sensor 240.

The controller 296 can further include a memory. The memory can be configured to store data received from the system 100. The memory can be implemented as a stand-alone memory unit and/or as part of the server 290 and/or the operating PC 292.

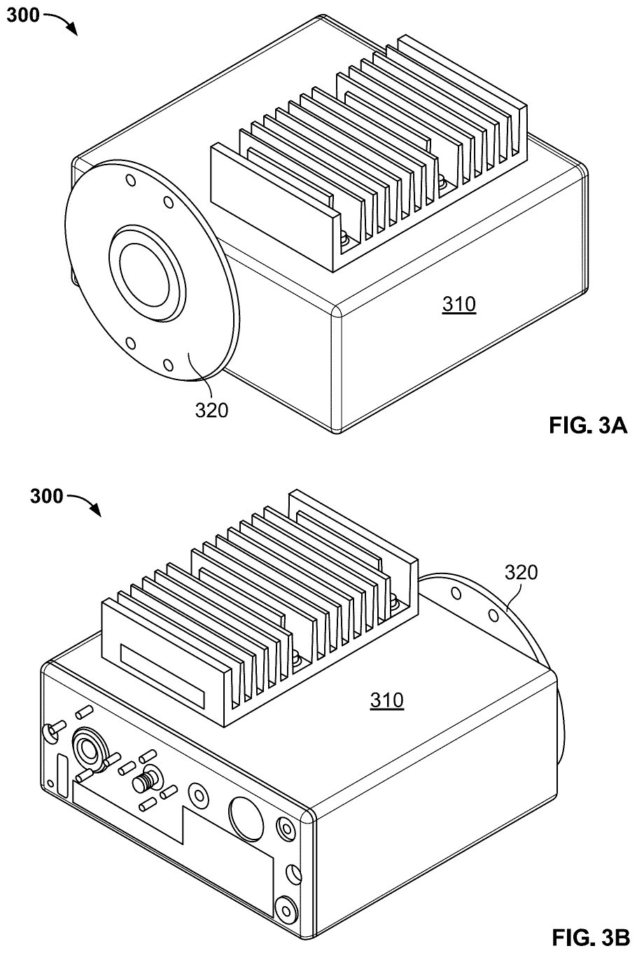

FIGS. 3A and 3B illustrate a front isometric view and a back isometric view of a sensor 300 according to one embodiment. The sensor 300 can be the first sensor 210 and the second sensor 240 of FIG. 2. The sensor 300 can be provided in the form of a near-infrared (NIR) spectrometry sensor. As shown, the sensor 300 can include a body 310 and a port 320. The sensor 300 can be installed in an anaerobic digester system, such as the system 100 of FIGS. 1 and 2, such that the port 320 forms a fluid path between the sensor 300 and the anaerobic digester system.

The sensor 300 can be configured to detect one or more physical and chemical properties of an anaerobic digester system such as TS, VS, COD, BOD, TKN, VFA, acetic acid, (FOG), FA, alkalinity, pH value, temperature, TSS, TDS, P₄, K, CO₂, O₂, CH₄, N₂, K₂O, P₂O₅, water flow rates, energy levels, organic loading, and relationships between these parameters. The sensor can be configured to detect one or more of the above parameters on a continuous basis and/or on a periodic basis, such as every 30 seconds, 60 seconds, 5 minutes, etc.

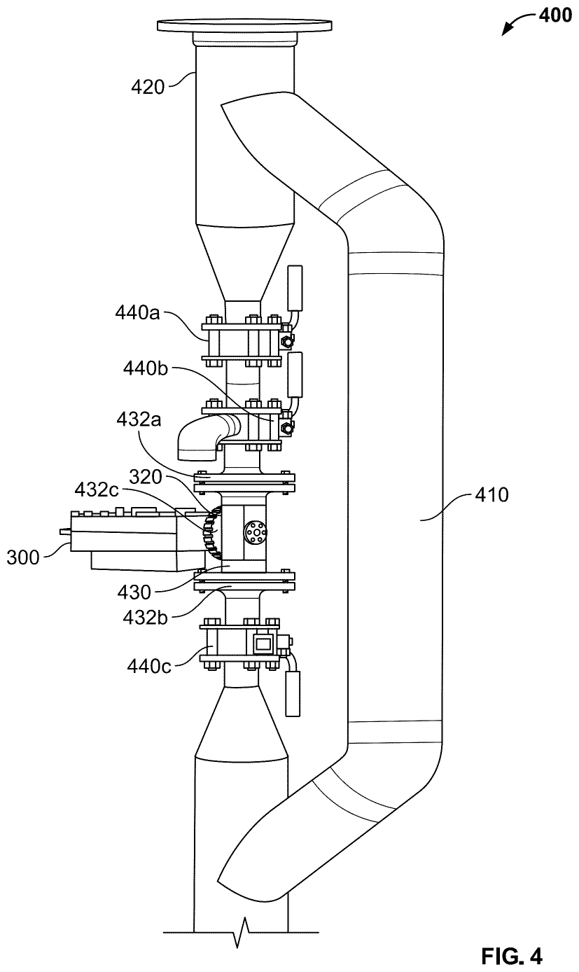

Turning to FIGS. 4-7B, several embodiments of sensor mountings are discussed. Referring first to FIG. 4, an isometric view of the sensor 300 of FIG. 3 installed in a bypass system 400 is illustrated. As shown, the bypass system 400 can include a fluid line 410 and a bypass line 420. The fluid line 410 can be an influent line, such as the feed line 120 of FIG. 1, an effluent line, such as the digestate product line 140 of FIG. 1, or any other fluid line included in an anaerobic digester system.

As shown, the port 320 of the sensor 300 can be connected to a bypass mounting 430. The bypass mounting 430 can include a first opening 432a, a second opening 432b, and a third opening 432c. The third opening 432c can form a fluid path between the bypass line 420 and the sensor 300. The bypass line 420 can include one or more valves for controlling the flow of fluid to the sensor 300. As shown, the bypass line includes a first valve 440a and, a second valve 440b upstream of the bypass mounting 430, and a third valve 440c downstream of the bypass mounting 430. Therefore, the sensor 300 can be isolated from the fluid line 410 without disrupting the operation of the anaerobic digester system.

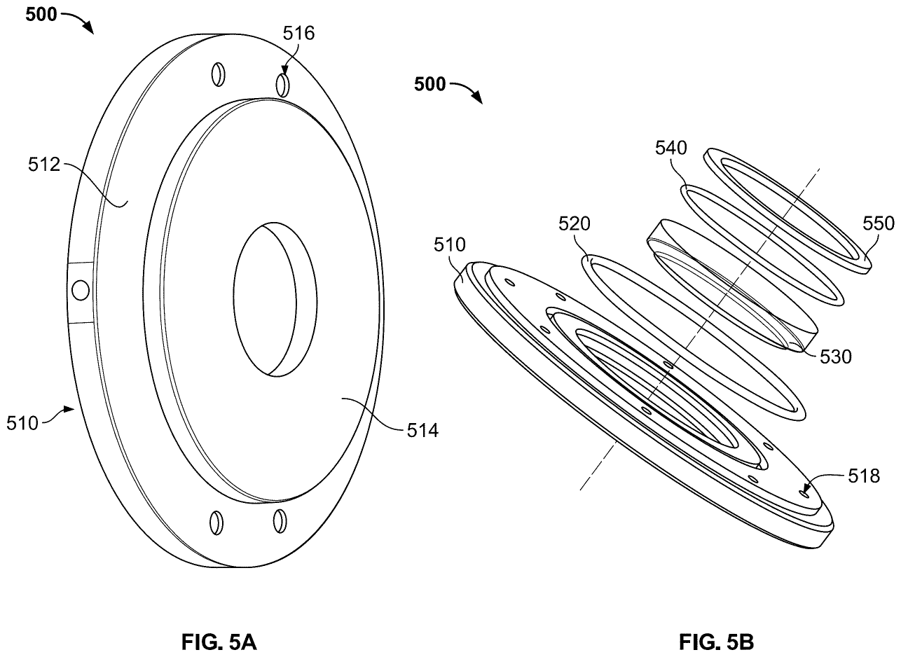

However, depending on the system structure or the desired location of the sensor, it can be ideal to place a sensor, such as the sensor 300, directly in-line and not on a bypass line of the anaerobic system. Thus, FIGS. 5-7B illustrate several example embodiments for weld-on flanges that can be used to mount a sensor to a fluid line or other components of the digester system.

Referring to FIGS. 5A and 5B, an isometric and exploded view, respectively, of a flat weld-on flange 500 for use with a sensor, such as the sensor 300 of FIG. 3, is illustrated. As shown, the flat weld-on flange 500 can be approximately disc-shaped. However, it is to be understood that the flat weld-on flange 500 can be provided in any shape known in the art. Regardless, the flat weld-on flange 500 can be ideally suited for relatively flat surfaces.

The flat weld-on flange 500 can include a body 510, including a first plate 512 and a second plate 514. As shown, the first plate 512 can include a first plurality of connection points 516 designed to serve as connection points for coupling the flat weld-on flange 500 to a pipe (e.g., first bypass line 220 and/or the second bypass line 250 of FIG. 2). Each connection point of the first plurality of connection points 516 can be designed to receive at least one coupling mechanism therein to couple the flat weld-on flange 500 to at least a portion of a pipe. For example, a coupling mechanism can be a screw or a nut and bolt.

Further, as shown best in FIG. 5B, the second plate 514 can include a second plurality of connection points 518 designed to serve as connection points between the flat weld-on flange 500 and a sensor, such as the sensor 300 of FIG. 3. The second plurality of connection points 518 can be similar to the first plurality of connection points 516.

The flat weld-on flange 500 can further include a first O-ring 520, a sapphire window 530, a second O-ring 540, and a gasket 550. The first O-ring 520 can be designed to provide a seal between the body 510 and the sapphire window 530. The second O-ring 540 and the gasket 550 can be designed to provide a seal between the sapphire window 530 and a sensor.

The sapphire window 530 can be a viewport or window-like structure designed to be transparent or semi-transparent. Thus, the NIR sensor can analyze the fluid in the pipe the flange is connected to without needing a liquid sample. A benefit to using a sapphire window 530 is that sapphire can be better suited for use in environments with temperatures, pressures, or compositions that would damage ordinary glass. Further, window or viewport flanges can be safer than conventional flanges because the window (e.g., the sapphire window 530) can act as a barrier between a process fluid (e.g., the fluid in the pipe the flange is connected to) and a user or operator. Accordingly, the sapphire window 530 can decrease the risk that the user or operator will be exposed to the process fluid.

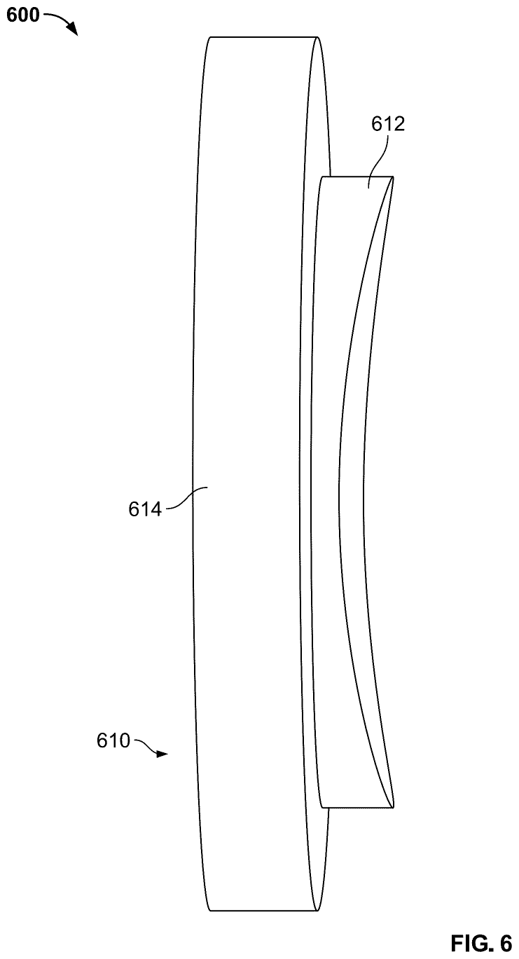

As mentioned above, the flat weld-on flange 500 can be best suited for installation on a relatively flat surface. However, it can be beneficial to have a curved flange, including a sapphire window 530, that can be installed directly onto a pipe, hopper, or other bent surface. Accordingly, FIG. 6 illustrates a side plan view of a curved weld-on flange 600.

The curved weld-on flange 600 can be similar to the flat weld-on flange 500. However, the curved weld-on flange 600 can include a body 610 having a first plate 612 and a second plate 614, where the first plate 612 is curved or concaved. The first plate 612 can be designed to be placed directly against a pipe or other curved surface, and a sensor (e.g., the sensor 300) can be mounted to the second plate 614. In some instances, the curvature of the first plate 612 can be designed to approximately match a curvature of the surface that the curved weld-on flange 600 is connected to.

Once installed on, the sensor (e.g. the sensor 300 of FIG. 3) can provide on-site monitoring of the anaerobic digester system without having to take field samples and/or run tests in a laboratory. As discussed above, the sensor can be configured to detect one or more physical and chemical properties. Further, the sensor can be communicatively coupled to a server configured to receive data from the sensor, interpret the data, store the data, and/or transmit the data.

FIG. 7 illustrates an exemplary monitoring system interface 700. The interface 700 can be displayed on a local or remote display, such as the operating PC 292 of FIG. 2. As shown, the interface 700 displays values for one or more biological parameters detected by a sensor on an influent line, such as the first sensor 210 positioned within the first bypass line 220 of the feed line 120 of FIG. 2. It is to be understood that the interface 700 can include more or fewer parameters depending on the embodiment. Further, in embodiments where the system includes multiple sensors, the interface 700 can include multiple displays, such as an influent display and an effluent display (i.e., values for one or more biological parameters detected by a sensor on an effluent line, such as the second sensor 240 positioned within the second bypass line 250 of the digestate product line 140.

A benefit of the interface 700 is that an operator can see data in near real time. Additionally, the sensor can improve safety because an operator can take fewer field samples. Therefore, the risk of exposure to hot and/or toxic samples can be decreased compared to traditional control methods that rely on field samples. Further, the monitoring system can be configured to determine if the one or more detected values are outside of a target range. The target range may, for example, be a predetermined target range. Upper and lower limits of the target range can be displayed on the interface 700. The interface 700 can be programmed to trigger an alert if the one or more detected values are outside of the target range. In other words, the interface 700 can display more or more alerts identifying out of compliance values. Thus, the interface 700 can alert an operator to a potential upset. In response to a parameter being above or below the upper and lower limits, an operator or a controller, such as the controller 296 of FIG. 2, can implement a corrective action.

For example, if the pH value in the anaerobic digester system is high, the interface 700 can display the pH value with an alert signal. The interface 700 may, for example, include an instruction for a corrective action to lower the pH value. Thus, the operator can acknowledge the alarm and perform the corrective action.

The interface 700 can display an alert for an out of compliance value and an indication that a controller has automatically adjusted one or more components of the anaerobic digester system. For example, in instances where the TS is above an upper TS limit, the controller can be designed to open or change a valve position of an effluent recycle valve and/or a make-up water valve. Accordingly, the interface 700 can include an indication or alert notifying the operator that the controller has adjusted one or more valves in response to the TS value being out of compliance.

The upper and lower limits of the target range for various physical or chemical parameters and example corrective actions are summarized in Table 1 below.

TABLE 1
System Parameter Limits and Potential Response Actions

| | | | Corrective Action | | | Corrective Action |
|---|---|---|---|---|---|---|
| | | | Taken to Bring | | | Taken to Bring |
| | | | Parameter Back | | | Parameter Back |
| | | | into Range Above | | | into Range Below |
| Parameter | Lower Limit | | Lower Limit | Upper Limit | | Upper Limit |
| Total Solids (TS) | 0.05% | | | 25% | | Recycling effluent, |
| | | | | | | or adding water |
| Volatile Solids | 85% of TS | | | 15% of TS | | |
| (VS) | | | | | | |
| Chemical-Oxygen | About equal to | | | About equal to | | |
| Demand (COD) | TBD of % TOC | | | TBD of % TOC | | |
| Biochemical- | 50% of COD mg/L | | | 85% of COD mg/L | | |
| Oxygen Demand | | | | | | |
| (BOD) | | | | | | |
| Total Organic | 35% of the total | | | 85% of the total V | | |
| Carbon* (TOC) | VS | | | | | |
| Total Kjeldahl | No less than 2% of | | | No less than 35% | | |
| Nitrogen (TKN) | TOC ppm, and/or | | | of TOC ppm, | | |
| | not to exceed 200 | | | and/or | | |
| | mg/l ammonium. | | | not to exceed 3,000 | | |
| | | | | mg/l ammonium. | | |
| Volatile Fatty | 0 | mg/L | Add Acetic Acid or | No higher than | | Add alkaline |
| Acids (VFA) | | | other VFA sources | 100% of BOD | | solution or Caustic |
| | | | | mg/L | | |
| Acetic Acid | Acetic acid is ⅓ | | | Acetic acid is ⅔ | | |
| | mg/L from total | | | mg/l from total | | |
| | VFA | | | VFA | | |
| Alkalinity | Effluent = 0 mg/L | | Add Caustic or | Effluent = .5 mg/L | | Add Muriatic Acid, |
| | Influent = 1:1 of | | Alkaline solution | Influent = 1:1 of | | or Acetic Acid |
| | Alkalinity:VFA | | | Alkalinity:VFA | | |
| pH Value | Influent = 3 | | Add Caustic | Influent = 8 | | Add acid solution |
| Temperature** | 60° | F. | | 160° | F. | |
| Total Suspended | +/−30% | | | +/−30% | | |
| Solids (TSS)*** | | | | | | |
| Total Dissolved | +/−30% | | | +/−30% | | |
| Solids (TDS) | | | | | | |
| Phosphate (P₄) | Nitrogen 10:1 mg/L | | | Nitrogen 2:1 mg/L | | |
| Potassium (K) | 300 | mg/L | | 12,000 | mg/L | |

| | | |
|---|---|---|
| FOS/TAC Ratio | Influent 1:1 | Influent 5:1 |
| | Effluent .1 | Effluent .6 |
| BOD/COD Ratio | 35% | 85% |

*TOC can be determined by the formula: ((% TS*% VS) = %*10,000 = mg/l)
**Temperature can also be monitored for fluctuations greater than about 1.0 degrees F. per day.
***% TS and % TDS can be determined by: TSS + TDS = ppm, where ppm*10,000 = % TS

It is to be understood that the above table is not to be considered exhaustive. Additional parameters such as FOG, FA, hydrogen sulfide, carbon dioxide, oxygen, methane, nitrogen, potassium oxide, phosphorus oxide, phosphorus pentoxide, TOC/TKN ratio, carbohydrates, lipids, water flow rate, and energy production rates can be monitored by the second sensor 240 and/or additional sensor.

Moreover, some metrics may not have set upper or lower limits but can be monitored for trends. Thus, the controller 296 can be designed to store historical data (e.g., historical detected values) for each of the system parameters discussed herein in the memory. The controller can then analyze the historical data to determine one or more system parameter trends. Further, in some instances, the controller 296 can be designed to predict potential upsets based on the one or more trends and/or comparing the detected values to the one or more trends.

For example, water and energy rates can have upper and lower limits and/or can be monitored for consistency, such that shifts to the system operations and health are monitored. When a shift in a trend is detected, the controller can be designed to instruct the interface 700 to display an alert that one or more parameters may be out of compliance with a trend.

Optionally, the controller can determine a detected value is out of compliance with a trend when the detected value is above or below a trend average by a threshold amount. The threshold amount can be different for each system parameter and its associated trend. Therefore, each trend can have a trend threshold range. Further, the trend threshold can be the target range for the associated trend.

For example, the detected value can be out of compliance with the trend average when the detected value is at least +/−10% of the average value for the trend (i.e., the trend threshold range is +/−10%). In another instance, the detected value can be out of compliance with the trend average when the detected value is at least +/−20% of the average value for the trend (i.e., the trend threshold range is +/−20%). In some aspects, the detected value can be out of compliance with the trend average when the detected value is at least +/−30% of the average value for the trend (i.e., the trend threshold range is +/−30%). In other instances, the detected value can be out of compliance with the trend average when the detected value is at least +/−40% of the average value for the trend (i.e., the trend threshold range is +/−40%).

It is to be further understood that the above ranges are estimates, and the potential response actions are not to be considered limiting. System limits and response actions can depend on the size, structure, feed stock, location, and needs of each particular system.

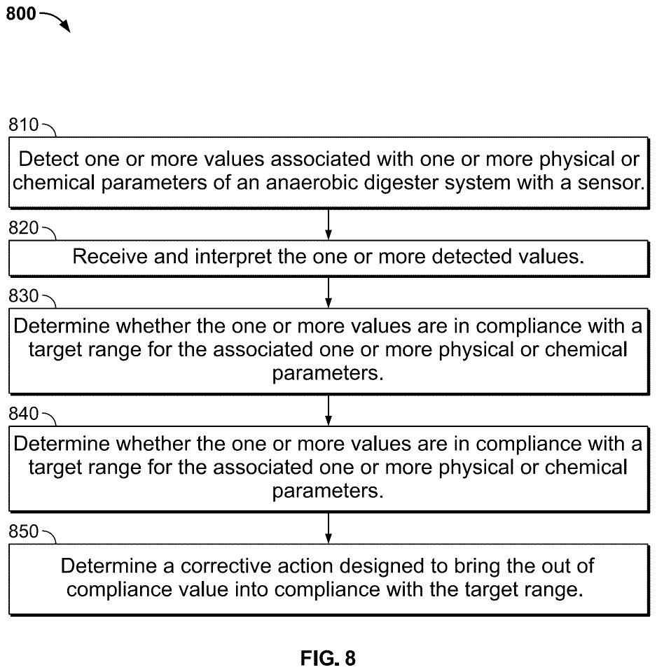

Also provided herein are methods of monitoring and controlling an anaerobic digester system. Accordingly, FIG. 8 illustrates a method 800 of controlling the anaerobic digester system 100 of FIG. 1.

At step 810, the controller 296 can be designed to detect one or more values associated with the one or more physical or chemical parameters of the anaerobic digester system 100. As discussed above, the one or more physical or chemical properties can include TS, VS, COD, BOD, COD, TKN, VFA, acetic acid, FOG, FA, alkalinity, pH value, temperature, TSS, TDS, H2S, P₄, K, K2O, P2O5, water flow rates, energy levels, and organic loading. The one or more physical or chemical parameters can be detected with the first sensor 210 and/or the second sensor 240.

At step 820, the server 290 can be designed to receive data (e.g., the detected one or more values) from the first sensor 210 and/or the second sensor 240, interpret the data, store the data (e.g., store the data as historical data), and/or transmit the data to the operating PC 292 and/or the controller 296.

At step 830, the controller 296 can be designed to determine whether the one or more detected values are in compliance with a target range for the associated one or more physical or chemical parameters of the detected values. For instance, as discussed above, some parameters can have a predetermined target range. In other instances, some parameters can have a target range that is based on a trend average and a target threshold range for the trend average.

At step 840, the controller 296 can be designed to determine whether the one or more values are in compliance with a target range for the associated one or more physical or chemical parameters. For example, the controller 296 can compare the detected one or more values to the target range for each value of the detected values. Accordingly, if the detected value is less than or greater than the target range, the detected value can be determined to be out of compliance with the target range. In some aspects, the controller 296 can be designed to trigger an alert indicating that one or more detected values are out of compliance. As mentioned above, in some instances, the alert can be displayed on an interface, such as the interface 700 of FIG. 7.

Optionally, at step 850, the controller 296 can be designed to determine a corrective action designed to bring the out of compliance value into compliance with the target range. For example, the corrective action can be one of the corrective actions described in Table 1 above. In some instances, the corrective action can be manually performed by an operator. In other instances, the controller 296 can be designed to automatically implement the corrective action. For example, the controller can be designed to adjust a valve position of one or more of the valves of the system 100 of FIG. 1 (e.g., the first valve 125, the second valve 135, the third valve 145, the fourth valve 155, the fifth valve 165, the sixth valve 175, and the seventh valve 185).

### EXAMPLES

The invention is now described with references to the following Examples. These Examples are provided with the purpose of illustration only, and the invention should in no way be construed as being limited to these Examples but rather should be construed to encompass any and all variations that become evident as a result of the teaching provided herein.

### Example 1

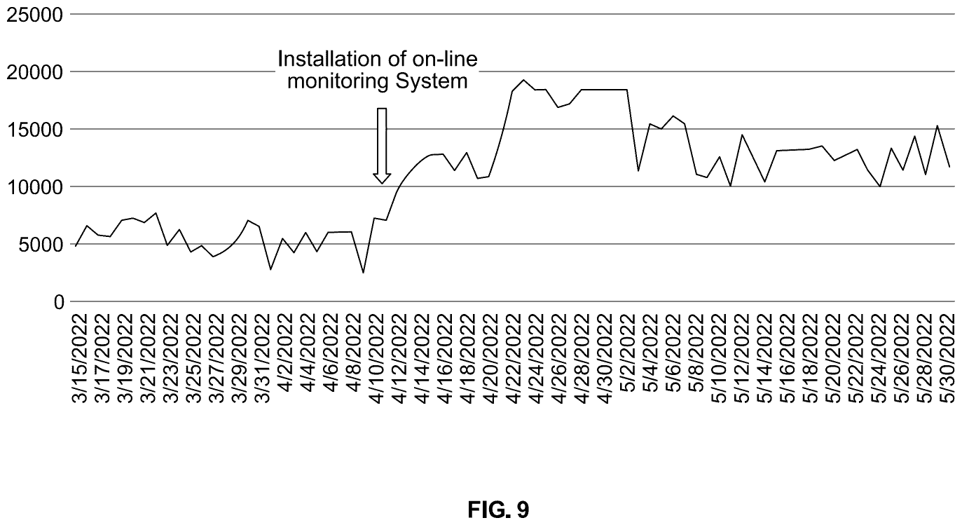

FIG. 9 is a graph showing increased energy production for an anaerobic digester system after the installation of an in-line monitoring system, as described above. As shown, prior to installation of the in-line monitoring system, the anaerobic digester system was producing about an average of 6,300 kilowatt-hours per day (kWh/Day). However, after installation and calibration of the in-line monitoring system, the anaerobic digester system was operating at about an average of 13,700 kWh/Day. Thus, the anaerobic digester system saw about a 217% increase in energy production. The increase in the energy production can be attributed to more consistent control of the digester system. Thus, the following examples include data from the same system and illustrate how the in-line monitoring system can help identify potential upset conditions which can then be addressed by adjusting one or more operating parameters of the system and/or treating the digester system with treatment chemistries.

### Example 2

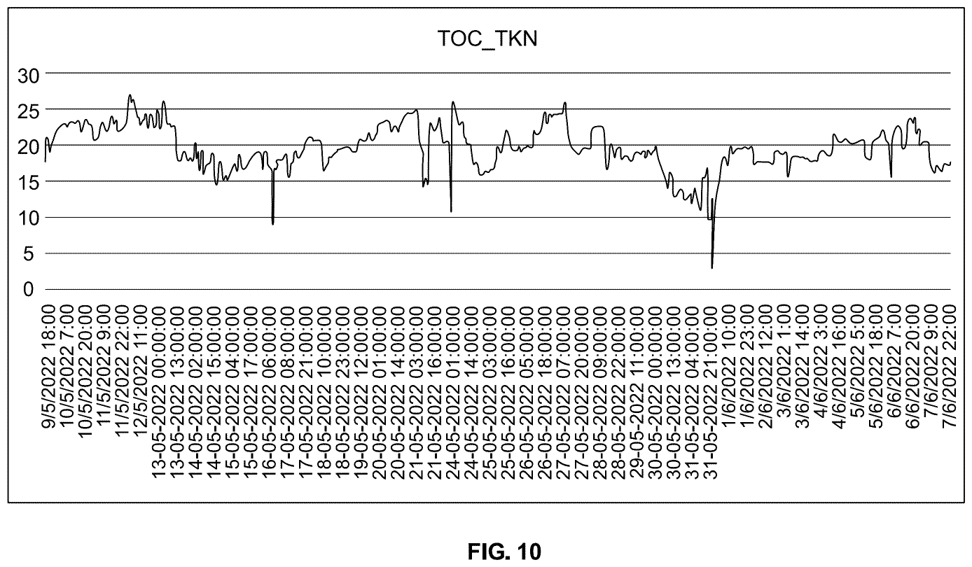

FIG. 10 is a graph showing a ratio between TOC and TKN levels for the anaerobic digester system of FIG. 9 after the installation of the in-line monitoring system. As shown, the anaerobic digester system generally operated at a desired carbon-to-nitrogen range of about 15:1 to 25:1. However, on May 31, 2022, due to a decrease in production, the anaerobic digester system experienced an imbalance in the carbon-to-nitrogen ratio. Without sufficient carbon in the system, the anaerobic digester system can experience nitrogen toxicity which can imbalance the micronutrients in the system and negatively impact performance. However, as shown, the carbon-to-nitrogen ratio was quickly brought back into the desired range because operations personnel were alerted to the imbalance in near real-time and were able to take corrective actions.

### Example 3

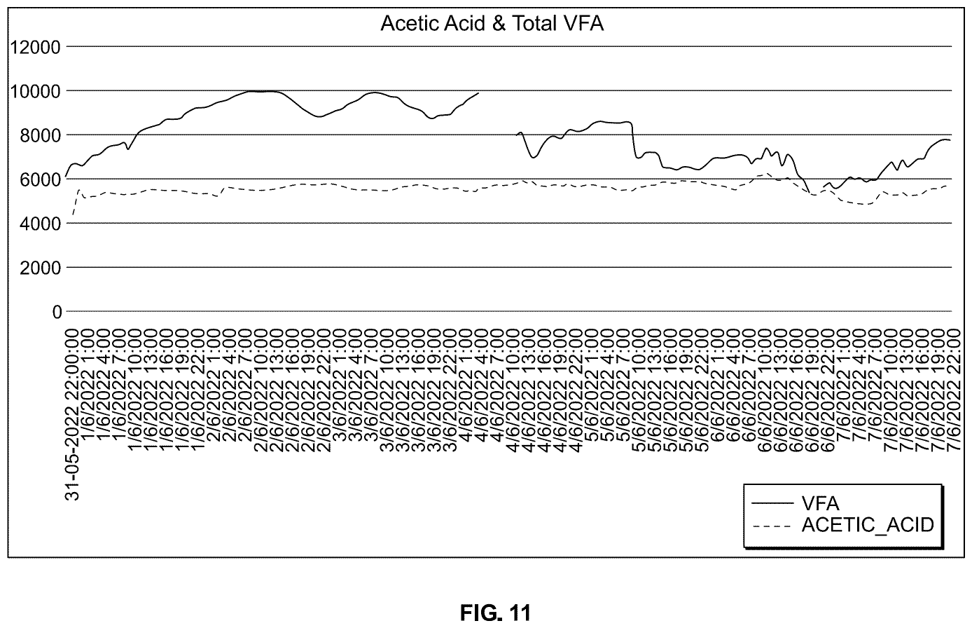

FIG. 11 is a graph showing acetic acid and total VFA levels for the anaerobic digester system of FIG. 9 after the installation of the in-line monitoring system. As shown, between approximately February 2022 and March 2022, VFA levels were between about 8,500 milligrams/L (mg/L) and 10,000 mg/L. VFAs in high concentrations can become toxic to the micronutrients in the system and can lower the anaerobic digester system pH value and contribute to a system upset. In conventional systems, VFAs are not measured daily. Thus, it can be difficult for operations personnel to catch high VFA levels. However, as shown, in an embodiment of the system disclosed herein a foaming event was prevented because operations personnel were able to monitor the VFA levels on at least a daily basis and adjust system parameters to avoid an upset.

Further, as shown on Jun. 6, 2022, the relationship between VFA and acetic acid levels were imbalanced. However, the imbalance was corrected by adding defoamer and micronutrients. Thus, the VFA and acetic acid levels were corrected within approximately one day. It is unlikely that a similar event would be controlled as quickly using traditional monitoring methods of taking field samples and sending them to a laboratory because the typical turnaround time for such methods is at least a day, if not more.

### Example 4

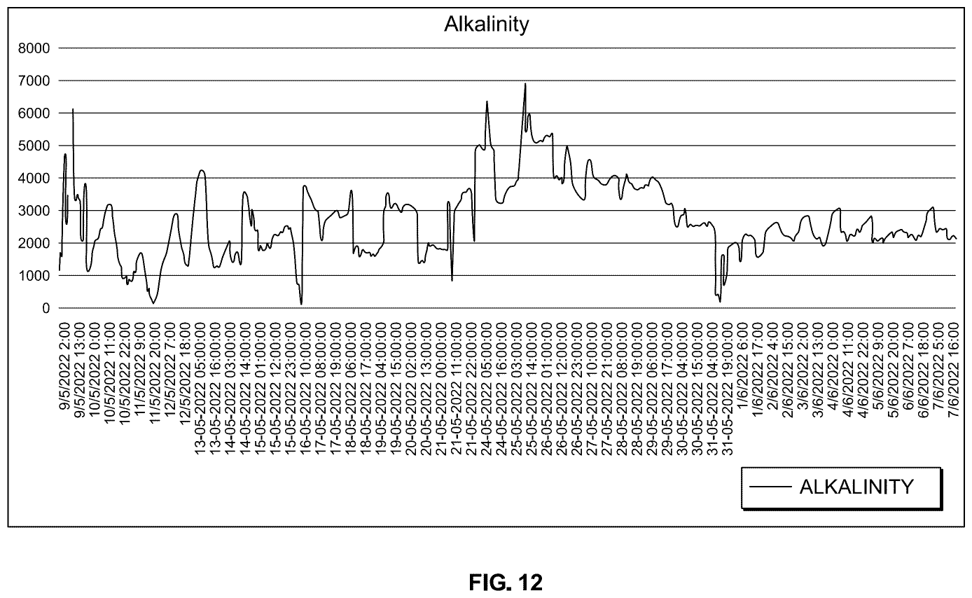

FIG. 12 is a graph showing alkalinity levels for the anaerobic digester system of FIG. 9 after the installation of the in-line monitoring system. As shown, between about May 21, 2022, to about May 31, 2022, alkalinity levels were imbalanced as compared to VFA levels (see also FIG. 11 showing VFA levels for the system). Alkalinity is a common indicator of the stability of the anaerobic digester system because it is the measure of the buffering capacity, or the ability to resist a change in pH value, due to the addition of acids or bases. Therefore, high levels of alkalinity can be an indication that VFAs and CO₂ levels are also high. Thus, operations personnel were able to avoid an upset because the in-line monitoring system alerted them to high alkalinity levels.

### Example 5

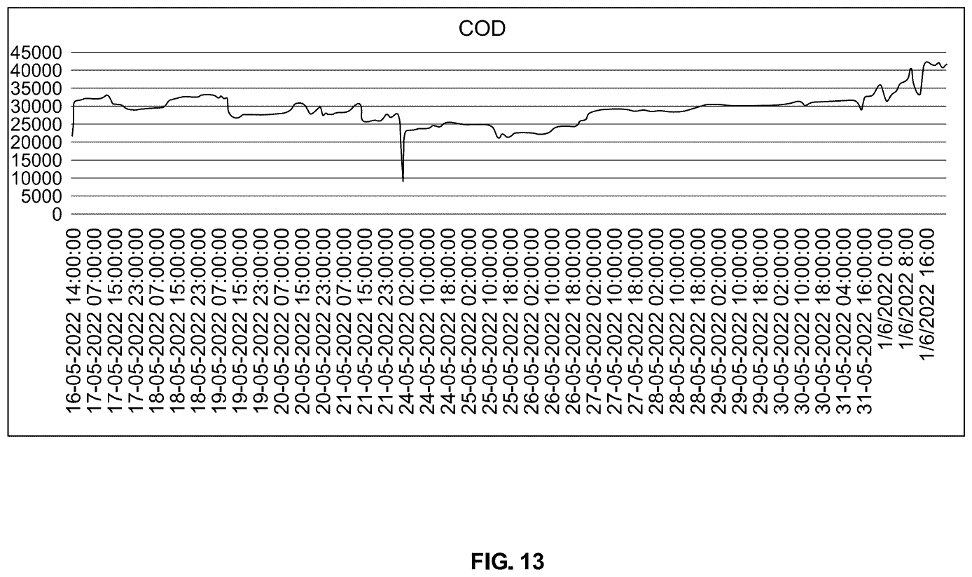

FIG. 13 is a graph showing COD levels for the anaerobic digester system of FIG. 9 after the installation of the in-line monitoring system. As shown, the COD demand gradually increased after May 2022. COD can be an indicator of methane production within the digester system. Thus, by determining COD demand several times a day, operations personnel can identify a potential imbalance in the digester system. Here, to correct the COD demand, the operations personnel were able to make changes to the feedstock input.

The above examples illustrate how consistent in-line monitoring can provide near real-time data on the condition of the anaerobic digester system. By having access to near real-time data, operations personnel can quickly identify potential issues and prevent or decrease downtime and upsets. Thus, a more consistent operation can improve safety and save money by keeping production high.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

1. An anaerobic digester monitoring system, comprising:
a first sensor in fluid communication with a first fluid line of an anaerobic digester system,
wherein the first sensor is designed to detect one or more values associated with one or more physical or chemical parameters of a first fluid in the first fluid line,
wherein the first sensor is communicatively coupled to an analyzer designed to transmit the one or more values to a server including a controller,
wherein the controller is designed to determine whether the one or more values are within a target range, and
wherein the server is communicatively coupled to an interface configured to display the one or more values and provide an indication that the one or more values are outside of the target range.

2. The anaerobic digester monitoring system of claim 1, wherein the first sensor is a near infrared (NIR) sensor.

3. The anaerobic digester monitoring system of claim 1 further comprising a second sensor in fluid communication with a second fluid line of the anaerobic digester system, wherein the second sensor is configured to detect one or more values associated with one or more physical or chemical parameters of a second fluid in the second fluid line.

4. The anaerobic digester monitoring system of claim 1, wherein the first fluid line is a feed line.

5. The anaerobic digester monitoring system of claim 3, wherein the second fluid line is a digestate line.

6. The anaerobic digester monitoring system of claim 1, wherein the one or more physical or chemical properties are selected from the group consisting of total solids (TS), volatile solids (VS), chemical oxygen demand (COD), biochemical oxygen demand (BOD), total organic carbon (COD), Total Kjeldahl Nitrogen (TKN), volatile fatty acids (VFA), acetic acid, fat, oil, and grease (FOG), fatty acids (FA), alkalinity, pH value, temperature, total suspended solids (TSS), total dissolved solids (TDS), hydrogen sulfide (H₂S), phosphorous (P₄), potassium (K), potassium oxide (K₂O), phosphorus pentoxide (P₂O₅), water flow rates, energy levels, and organic loading.

7. An anaerobic digester system, comprising:
a main reactor;
an influent line in fluid communication with the main reactor;
an effluent line in fluid communication with the main reactor; and
a monitoring system comprising:
a first sensor in fluid communication with the influent line, the first sensor designed to detect one or more values associated with one or more physical or chemical parameters of a fluid in the influent line; and
an analyzer communicatively coupled to the first sensor and a server including a controller,
wherein the server is designed to:
receive the one or more values;
interpret the one or more values; and
transmit the one or more values to an interface configured to display the interpreted one or more values.

8. The anaerobic digester system of claim 7, wherein the server is designed to determine whether each value of the one or more values is out of compliance with a target range.

9. The anaerobic digester system of claim 8, wherein the interface is designed to display an alert in response to the controller determining that at least one value of the one or more values is out of compliance with the target range.

10. The anaerobic digester system of claim 8, wherein the controller is designed to determine a corrective action designed to bring the one or more out of compliance values into compliance with the target range.

11. The anaerobic digester system of claim 10, wherein the controller is designed to automatically implement the corrective action.

12. The anaerobic digester system of claim 7, wherein the controller is designed to:
store a plurality of historical values associated with the one or more physical or chemical parameters in a memory;
determine a trend based on the plurality of historical values;
determine a trend average of the trend;
determine a target range based on the trend average; and
determine whether the one or more values are greater than or less than the target range.

13. A method for controlling an anaerobic digester system, the method comprising:
detecting one or more values associated with one or more physical or chemical parameters of a first fluid line of the anaerobic digester system with a first sensor;
interpreting the one or more values; and
transmitting the one or more values to an interface designed to display the one or more values.

14. The method of claim 13, further comprising detecting one or more values associated with one or more physical or chemical parameters of a second fluid line of the anaerobic digester system with a second sensor.

15. The method of claim 13, further comprising determining whether each value of the one or more values is within a target range.

16. The method of claim 15, further comprising displaying an alert on the interface in response to determining at least one value of the one or more values is not within the target range.

17. The method of claim 16, further comprising determining a corrective action designed to bring the at least one value within the target range.

18. The method of claim 13, further comprising storing a plurality of historical values associated with one or more physical or chemical parameters in a memory of a controller, and determining a trend based on the plurality of historical values.

19. The method of claim 18, further comprising determining a trend average of the trend, and determining a trend threshold range based at least in part on the trend average.

20. The method of claim 19, further comprising determining whether at least one value of the one or more values is within the trend threshold range.
